(19) United States
(12) Patent Application Publication
(10) Pub. No.: US 2005/0053600 A1
(43) Pub. Date: Mar. 10, 2005

(54) Methods for treating rheumatoid arthritis

(76) Inventor: Thomas E. Lane, Irvine, CA (US)

Correspondence Address: O'MELVENY & MEYERS, 114 PACIFICA, SUITE 100, IRVINE, CA, 92618, US

(21) Appl. No.: 10/938,673

(22) Filed: Sep. 9, 2004

Related U.S. Application Data
(60) Provisional application No. 60/501,312, filed on Sep. 9, 2003.

(51) Int. Cl. A61K 39/395

(52) U.S. Cl. 424/141.1; 424/146.1

(57) ABSTRACT

The invention discloses methods and compositions for treating rheumatoid arthritis through the administration of humanized anti-IP-10 antibody alone or in combination with an additional anti-rheumatic therapeutic compound.

### RELATED APPLICATION INFORMATION

This application claims the benefit of U.S. Provisional Application No. 60/501,312, filed Sep. 9, 2003.

### FIELD OF THE INVENTION

The present invention relates generally to compositions and methods for treating autoimmune disorders, especially rheumatoid arthritis (RA), by selective inhibition of the chemokine IP-10. Specifically, the invention relates to use of a therapeutically effective amount of a humanized anti-IP-10 antibody, optionally in combination with another anti-RA therapeutic compound to treat RA.

### BACKGROUND OF THE INVENTION

Rheumatoid arthritis (RA) is a chronic inflammatory disease of unknown etiology affecting diarthrodial joints. The disease is characterized by three main manifestations, namely inflammation, abnormal cellular and humoral immunoresponse, and synovial hyperplasia. Eventually the interplay between these pathologic processes can lead to complete joint destruction. (Gay, S., et al. 1993. Molecular and cellular mechanisms of joint destruction in rheumatoid arthritis: two cellular mechanisms explain joint destruction? Ann. Rheum. Dis., 52:39-47).

A hallmark of RA is infiltration of leukocytes into synovial tissue, mediated by a complex network of cytokines, adhesion molecules and chemoattractants. (Kunkel, S. L. 1999. Through the looking glass: the diverse in vivo activities of chemokines. J. Clin. Invest., 104:1333-1334). The presence of activated leukocytes contributes to persistence of destructive synovitis. (Murdoch, C., Finn, A., 2000. Chemokine receptors and their role in inflammation and infectious diseases. Blood, 95:3032-3043).

Leukocyte recruitment to the affected joint is not yet well understood. (Ruschpler, P., et al. 2003. High CXCR3 expression in synovial mast cells associated with CXCL9 and CXCL10 expression in inflammatory synovial tissues of patients with rheumatoid arthritis. Arthritis. Res. Ther. 2003 5:R241-R252). Macrophages are major components in the inflammatory cascade (Burmester, G. R., et al. 1997. Mononuclear phagocytes and rheumatoid synovitis. Mastermind or workhorse in arthritis? Arthritis Rheum., 40:5-18) and are also believed to be important mediators of joint destruction. (Scott, B. B., et al. 1997. Rheumatoid arthritis synovial fibroblast and U937 macrophage/monocyte cell line interaction in cartilage degradation. Arthritis Rheum., 40:490-498). Large numbers of macrophages are present in synovial tissue (Lindblad, S., et al. 1983. Phenotypic characterization of synovial tissue cells in situ in different types of synovitis. Arthritis Rheum., 26:1321-1332) and their cell numbers are associated with scores for local disease activity in clinically involved joints of RA patients. (Tak, P. P., et al. 1997. Analysis of the synovial cellular infiltrate in early rheumatoid synovial tissue in relation to disease activity. Arthritis Rheum. 40:217-225). Macrophage activity in the synovial compartment includes the production of chemotactic cytokines known as chemokines (Kunkel, S. L., et al. 1996. The role of chemokines in inflammatory joint disease. J. Leukoc. Biol. 59:6-12), but the role and effect of these chemokines on the progression of RA remains to be determined.

Chemokines can be generally divided into four groups: the CXC, C, CX₃C and CC chemokine receptor families. The CXC subfamily includes CXCL-8 [interleukin (IL)-8], CXCL-9 [monokine induced by γ-interferon (Mig)], and CXCL10 [interferon-γ inducible protein-10 (IP-10)]. CXC chemokines are mostly chemotactic factors for neutrophils, although CXCL10 and CXCL9 attract monocytes and T lymphocytes. The CC chemokines include CCL2 [monocyte chemoattractant protein 1] and CCL4 [macrophage-inhibiting protein-1β (MIP-1β)]. The main function of both CCL2 and CCL4 seems to be the recruitment of macrophages. The complex interaction of these chemokines in RA is yet to be elucidated.

There remains a need to identify suitable therapeutic targets that can be used for the treatment of rheumatoid arthritis, and to develop therapeutic agents which modulate such a target.

### SUMMARY OF INVENTION

The present invention provides a method for treating rheumatoid arthritis in an individual comprising administering to the individual a therapeutically effective amount of humanized anti-IP-10 antibody. Optionally, the humanized anti-IP-10 antibody is administered in combination with a therapeutically effective amount of another anti-RA therapeutic compound selected from the group of anti-inflammatory agents, disease-modifying-anti-rheumatic drugs, anti-rheumatic biologicals, and immune suppressors. In one embodiment, the anti-inflammatory agents may be aspirin, NSAIDS, coxibs, and corticosteriods. In another embodiment, the disease-modifying-anti-rheumatic drugs may be methotrexate, injectable gold, oral gold, hydroxychloroquine, and sulfasalazine. In yet another embodiment, the anti-rheumatic biologicals may be etanercept, infliximab, leflunomide, and anakinra. In yet another embodiment, the immune suppressors may be azathioprine or cyclophosphamide. In yet another embodiment, the humanized anti-IP-10 antibody may be administered in combination with a therapeutically effective amount of a compound listed in Table 2.

Also provided is a kit containing humanized anti-IP-10 antibody packaged alone or together with an anti-RA therapeutic compound candidate. The kit may be packaged with instructions for selection of an appropriate anti-RA therapeutic compound, as well as instructions for use and proper dosing.

### BRIEF DESCRIPTION OF THE DRAWINGS

FIG. 1 shows representative paws after treatment according to the methods of the present invention. A. Negative control; B. Immediate Anti-1-10; C. Positive Control

FIG. 2 illustrates that the methods of the present invention reduce inflammation. Square boxes=Control; Diamonds aCCL5 (anti-RANTES antibody); Circles=aIP-10 late (anti-IP-10 antibody, late treatment); Triangles=aiP-10 early (anti-IP-10 antibody, early treatment).

FIG. 3 illustrates measurements of paw inflammation. Square boxes=negative (N=3); Diamonds=No Treatment (N=4); Circles=Immediate anti-IP-10 antibody treatment (N=2); Triangles=Late anti-IP-10 antibody treatment (N=3)

FIG. 4 records histological scoring of inflammation.

### DETAILED DESCRIPTION OF THE INVENTION

The present invention provides a method for treating rheumatoid arthritis in an individual comprising administering to the individual a therapeutically effective amount of humanized anti-IP-10 antibody. Optionally, the humanized anti-IP-10 antibody is administered in combination with a therapeutically effective amount of an anti-RA therapeutic compound.

The present invention provides, for the first time, evidence that administering an anti-IP-10 antibody successfully reduces inflammation in the Type II Collagen-induced arthritis mouse model, which is an accepted model of human inflammatory and arthritic diseases.

As used herein, “anti-IP-10 antibody” encompasses antibodies and antigen binding fragments thereof that have specific binding affinity for the chemokine IP-10. This definition includes, but is not limited to antigen binding fragments such as Fab, F(ab′)₂, Fc, Fd and Fv fragments and the like, non-naturally occurring antibodies, such as, for example, single chain antibodies, chimeric antibodies, bifunctional antibodies, complementarity determining region-grafted (CDR-grafted) antibodies and humanized antibodies. Furthermore, the definition includes but is not limited to both polyclonal, monoclonal, humanized, or primatized antibodies against IP-10. Other aspects of the definition of “anti-IP-10 antibody” are described elsewhere below.

A description of the role and effect of anti-IP-10 antibody in the treatment of demyelinating diseases, such as Multiple Sclerosis, and remyelination, such as spinal cord injury, was first set out in PCT publications WO 02/15932 and WO 03/06045, respectively, both incorporated herein by reference. The chemokine IP-10, described at length therein, is also known as CXCL10, interferon inducible protein of 10 kDa, Small inducible cytokine B10 (Precursor), Interferon-gamma induced protein, and Gamma-IP10. IP-10 is the product of the gene alternately known as CXCL10, SCYB10, and INP10.

Details on the role of chemokines in disease processes, and the importance of modulating chemokine networks in the treatment of various diseases are reviewed in Schwarz, MK and Wells, TNC. 2002. Nature Reviews Drug Discovery. 1:347-358. Small molecule antagonists of CXCR3 (the major receptor for IP-10) have been suggested to be useful for the treatment of RA (see, for example, WO 02/85861 Kyowa/Millenium; WO 02/83143 Tularik; WO 02/72549 Millenium/Warner-Lambert; WO 02/85862 Kyowa/Millenium; WO 01/16114 Chemocentryx; and WO 03/24404 Schering AG).

As used herein, “rheumatoid arthritis” refers to a recognized disease state which may be diagnosed according to the 2000 revised American Rheumatoid Association criteria for the classification of rheumatoid arthritis, or any similar criteria. Physiological indicators of RA include, symmetric joint swelling which is characteristic though not invariable in rheumatoid arthritis. Fusiform swelling of the proximal interphalangeal (PIP) joints of the hands as well as metacarpophalangeal (MCP), wrists, elbows, knees, ankles and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension or hyperflexion of the MCP and PIP joints, flexion contractures of the elbows, and subluxation of the carpal bones and toes.

As used herein, the term “treating rheumatoid arthritis” refers to a decrease in severity of RA symptoms, an increase in frequency and duration of RA symptom-free periods, or a prevention of impairment or disability due to RA affliction. Clinically, successful RA treatment may be measured by preventing further deterioration of physical symptoms associated with RA, such as, for example, pain, fatigue, morning stiffness (lasting more than one hour), diffuse muscular aches, loss of appetite, weakness, joint pain with warmth, swelling, tenderness, and stiffness of a joint after inactivity. Treating rheumatoid arthritis also includes preventing or delaying onset of RA, such as may be desired when early or preliminary signs of the disease are present. Likewise it includes delaying chronic progression associated with RA. Laboratory test utilized in the diagnosis of RA include chemistries (including the measurement of IP-10 levels), hematology, serology and radiology. Accordingly, any clinical or biochemical assay that monitors any of the foregoing may be used to determine whether a particular treatment is efficacious and successful for treating RA in an individual following treatment of the individual with the methods of the present invention.

Preparation of Anti-IP-10 Antibody

Methods of preparing and isolating antibodies, including polyclonal and monoclonal antibodies, using peptide immunogens, are well known to those skilled in the art and are described, for example, in Harlow and Lane, Antibodies: A Laboratory Manual, Cold Spring Harbor Laboratory Press (1988). Non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Hoogenboom et al., U.S. Pat. No. 5,564,332, issued Oct. 15, 1996; Winter and Harris, Immunol. Today 14:243-246 (1993); Ward et al., Nature 341:544-546 (1989); Harlow and Lane, Antibodies. A laboratory manual (Cold Spring Harbor Laboratory Press, 1988); Hilyard et al., Protein Engineering: A Practical Approach (IRL Press 1992); Borrabeck, Antibody Engineering, 2 d ed. (Oxford University Press 1995); each of which is incorporated herein by reference).

An IP-10 specific antibody can be raised using as an immunogen a substantially purified IP-10 protein, which can be prepared from natural sources or produced recombinantly. Additionally, a peptide portion of an IP-10 protein, including synthetic peptides, may be used as an immunogen for an IP-10 specific antibody. Moreover, a non-immunogenic peptide portion of a IP-10 protein may be made immunogenic by coupling the hapten to a carrier molecule, such as bovine serum albumin or keyhole limpet hemocyanin, or by expressing the peptide portion as a fusion protein. Various other carrier molecules and methods for coupling a hapten to a carrier molecule are well known in the art (see Harlow and Lane, supra, 1988; see, also, Hermanson, Bioconjugate Techniques, Academic Press, 1996, which is incorporated herein by reference).

An anti-IP-10 antibody can be labeled so as to be detectable, or for therapeutic purposes, using methods well known in the art (Hermanson, supra, 1996; Harlow and Lane, supra, 1988; 5 chap. 9). For example, an IP-10 antibody can be linked to a radioisotope, fluorophore, or other detectable agent by methods well known in the art.

Methods for raising polyclonal antibodies, for example, in a rabbit, goat, mouse or other mammal, are well known in the art (Harlow and Lane, supra, 1988). The production of anti-peptide antibodies commonly involves the use of host animals such as rabbits, mice, guinea pigs, or rats. If a large amount of serum is needed, larger animals such as sheep, goats, horses, pigs, or donkeys can be used. Animals are usually chosen based on the amount of antiserum required and suitable animals include rabbits, mice, rats, guinea pigs, and hamsters. These animals yield a range of 1-25 mL per single bleed (Harlow and Lane, infra, 1988). Rabbits are very useful for the production of polyclonal antisera, since they can be safely and repeatedly bled and produce high volumes of antiserum. Two injections two to four weeks apart with 15-50 pg of antigen in a suitable adjuvant such as, for example, Freund's Complete Adjuvant can be followed by blood collection and analysis of the antiserum.

In addition, monoclonal antibodies can be obtained using methods that are well known and routine in the art (Harlow and Lane, supra, 1988). A peptide portion of a protein such as IP-10 for use as an immunogen can be determined by methods well known in the art. Spleen cells from an IP-10 immunized mouse can be fused to an appropriate myeloma cell line to produce hybridoma cells. Cloned hybridoma cell lines can be screened using a labeled IP-10 protein to identify clones that secrete anti-IP-10 antibodies. Hybridomas expressing anti-IP10 monoclonal antibodies having a desirable specificity and affinity can be isolated and utilized as a continuous source.

Humanized antibodies can be constructed by conferring essentially any antigen binding specificity onto a human antibody framework. Methods of constructing humanized antibodies are useful to prepare an antibody appropriate for practicing the methods of the invention and avoiding host immune responses against the antibody when used therapeutically. Current leaders in this field are Medarex, Inc. (Princeton, N.J.), Abgenix, Inc. (Freemont, Calif.) and Protein Design Labs, Inc (Fremont, Calif.).

The antibody described above can be used to generate human therapeutic agents by methods well known in the art such as complementary determining region (CDR)-grafting and optimization of framework and CDR residues. For example, humanization of an antibody can be accomplished by CDR-grafting as described in Fiorentini et al., Immunotechnology 3(1):45-59 (1997), which is incorporated herein by reference. Briefly, CDR-grafting involves recombinantly splicing CDRs from a non-human antibody into a human framework region to confer binding activity onto the resultant grafted antibody, or variable region binding fragment thereof.

Once the CDR-grafted antibody, or variable region binding fragment is made, binding affinity comparable to the non-human antibody can be reacquired by subsequent rounds of affinity maturation strategies known in the art. Humanization of antibody in the form of rabbit polyclonal antibodies can be accomplished by similar methods as described in Rader et al., J. Biol. Chem. 275(18):13668-13676 (2000), which is incorporated herein by reference.

Humanization of a non-human anti-IP-10 antibody can also be achieved by simultaneous optimization of framework and CDR residues, which permits the rapid identification of co-operatively interacting framework and CDR residues, as described in Wu et al., J. Mol. Biol. 294(1):151-162 (1999), which is incorporated herein by reference. Briefly, a combinatorial library that examines a number of potentially important framework positions is expressed concomitantly with focused CDR libraries consisting of variants containing random single amino acid mutations in the third CDR of the heavy and light chains. By this method, multiple Fab variants containing as few as one non-human framework residue and displaying up to approximately 500-fold higher affinity than the initial chimeric Fab can be identified. Screening of combinatorial framework-CDR libraries permits identification of monoclonal antibodies with structures optimized for function, including instances in which the antigen induces conformational changes in the monoclonal antibody. The enhanced humanized variants contain fewer non-human framework residues than antibodies humanized by sequential in vitro humanization and affinity maturation strategies known in the art.

It is further contemplated that an anti-IP-10 antibody of the invention can be a human antibody or a primatized antibody. In addition, human antibodies can be produced by methods known in the art that involve immunizing a transgenic non-human animal with the desired antigen. The transgenic non-human animal can be modified such that it fails to produce endogenous antibodies, but instead produces B-cells which secrete fully human immunoglobulins. The antibodies produced can be obtained from the animal directly or from immortalized B-cells derived from the transgenic non-human animal.

As used herein a “humanized antibody” is one that contains up to 100% human protein sequences. Humanized antibodies may be derived from other organisms, such as another mammal or vertebrate, but one or more specific amino acids of the antibody are changed, by any technique known in the art, so as to conform more closely to an antibody that would typically be generated in a human or primate. Humanized antibodies may also be generated from rodents, such as transgenic mice carrying a suite of human immunological genes. Humanized antibodies are anticipated to result in a favorable safety profile which are also likely to be eliminated less rapidly from the human body, potentially reducing dosage frequency and amount.

Alternatively, the genes encoding the immunoglobulins with human variable regions can be recovered and expressed to obtain the antibodies directly or modified to obtain analogs of antibodies such as, for example, single chain Fv molecules. Thus, it is contemplated to produce an anti-IP-10 antibody of the invention that is a fully human immunoglobulin to the IP-10 antigen or to produce an analog of the immunoglobulin by a process that includes immunizing a non-human animal with antigen under conditions that stimulate an immune response.

The non-human animal that produces a human antibody of the invention can be modified to be substantially incapable of producing endogenous heavy or light immunoglobulin chain, but capable of producing immunoglobulins with both human variable and constant regions. In the resulting immune response, the animal produces B cells which secrete immunoglobulins that are fully human and specific for the antigen. The human immunoglobulin of desired specificity can be directly recovered from the animal, for example, from the serum, or primary B cells can be obtained from the animal and immortalized. The immortalized B cells can be used directly as the source of human antibodies or, alternatively, the genes encoding the antibodies can be prepared from the immortalized B cells or from primary B cells of the blood or lymphoid tissue, for example, spleen, tonsils, lymph nodes, bone marrow, of the immunized animal and expressed in recombinant hosts, with or without modifications, to produce the immunoglobulin or its analogs. In addition, the genes encoding the repertoire of immunoglobulins produced by the immunized animal can be used to generate a library of immunoglobulins to permit screening for those variable regions which provide the desired affinity. Clones from the library which have the desired characteristics can then be used as a source of nucleotide sequences encoding the desired variable regions for further manipulation to generate human antibodies or analogs with these characteristics using standard recombinant techniques.

Various techniques for preparing human antibodies using transgenic non-human animals, for example, transgenic mice, are well known in the art and described, for example, in Fishwild et al., Nature Biotech. 14:845-851 (1996); Heijnen et al., J. Clin. Invest. 97:331-338 (1996); Lonberg et al. Nature 368:856-859 (1994); Morrison, Nature 368:812-813 (1994); Neuberger, Nature Biotech. 14:826 (1996); Chadd and Chamow, Curr. Opin. Biotechnol. 12(2):188-94 (2001); Russel et al., Infection and Immunity 1820-1826 (2000); Gallo et al., E. J. Immunol. 30:534-540 (2000); Davis et al., Cancer Metastasis Rev. 18(4):421-5 (1999); Green, J. Immunol. Meth. 231:11-23 (1999); Yang et al., J. Leukocyte Biol. 66:401-410 (1999); Jakobovits, Advanced Drug Del. Rev. 31:33-42 (1998); Green and Jakobovits, J. Exp. Med. 188(3):483-495 (1998); Jakobovits, Exp. Opin. Invest. Drugs 7(4):607-614 (1998); Mendez et al., Nature Genetics 15:146-156 (1997); Jakobovits, Weir's Handbook of Experimental Immunology, The Integrated Immune System, Vol. IV: 194 194.7 (1996), each of which is incorporated herein by reference. Furthermore, various techniques known in the art for preparation of a human antibody are described in U.S. Pat. Nos. 6,162,963; 6,150,584; 6,114,598; 6,111,166; 6,096,311 and 6,075,181, each of which is incorporated herein by reference.

Another highly efficient means for generating recombinant antibodies is by primatization as described by Newman, Biotechnology 10:1455-1460 (1992), which is incorporated herein by reference. More particularly, primatized antibodies can be generated, which antibodies contain monkey variable domains and human constant sequences.

Methods for primatization are known in the art and described in U.S. Pat. No. 6,113,898, which is incorporated herein by reference. Antibodies can be primatized such that they are not antigenically rejected upon human administration. Primatization relies on immunization of primates, for example, cynomolgus monkeys (Macaca fascicularis), with human antigens or receptors and can be used to generate high affinity monoclonal antibodies directed to human cell surface antigens.

Antibodies generated by primatization have previously been reported to display human effector function, have reduced immunogenicity, and long serum half-life and are thus useful as therapeutic agents of the present invention. The technology relies on the fact that despite the fact that cynomolgus monkeys are phylogenetically similar to humans, they still recognize many human proteins as foreign and therefore mount an immune response.

Moreover, because the cynomolgus monkeys are phylogenetically close to humans, the antibodies generated in these monkeys have been discovered to have a high degree of amino acid homology to those produced in humans. Indeed, after sequencing macaque immunoglobulin light and heavy chain variable region genes, it was found that the sequence of each gene family was 85-98% homologous to its human counterpart (Newman et al., supra, 1992). The first antibody generated by primatization, an anti-CD4 antibody, was 91-92% homologous to the consensus sequence of human immunoglobulin framework regions (Newman et al., supra, 1992). Methods known in the art for generation of a primatized antibody and useful for preparing an anti-IP-10 antibody of the invention further are described by Newman et al., Clin. Immunol. 98(2):164-74(2001); and Reddy et al., J. Immunol. 164(4):1925-33 (2000), both of which are incorporated herein.

As described above, anti-IP-10 antibodies of the invention include, for example, polyclonal antibodies, monoclonal antibodies, as well as recombinant versions and functional fragments thereof. Recombinant versions of these antibodies include a wide variety of constructions ranging from simple expression and co-assembly of encoding heavy and light chain cDNAs to specialty constructs termed designer antibodies. Recombinant methodologies, combined with the extensive characterization of polypeptides within the immunoglobulin superfamily, and particularly antibodies, provides the ability to design and construct a vast number of different types, styles and specificities of binding molecules derived from immunoglobulin variable and constant region binding domains. Specific examples include chimeric antibodies, where the constant region of one antibody is substituted with that of another antibody, and humanized antibodies, described above, where the complementarity determining regions (CDR) from one antibody are substituted with those from another antibody.

Other recombinant versions of antibody include, for example, functional antibody variants where the variable region binding domain or functional fragments responsible for maintaining antigen binding is fused to an Fc receptor binding domain from the antibody constant region. Such variants are essentially truncated forms of antibodies that remove regions non-essential for antigen and Fc receptor binding. Truncated variants can have single valency, for example, or alternatively be constructed with multiple valencies depending on the application and need of the user. Additionally, linkers or spacers can be inserted between the antigen and Fc receptor binding domains to optimize binding activity as well as contain additional functional domains fused or attached to effect biological functions other than IP-10 neutralization. Those skilled in the art will know how to construct recombinant antibodies specific for IP-10 in light of the art knowledge regarding antibody engineering and given the guidance and teachings herein. A description of recombinant antibodies, functional fragments and variants and antibody-like molecules can be found, for example, in “Antibody Engineering,” 2nd Edition, (Carl A. K. Borrebaeck, Ed.) Oxford University Press, New York, (1995).

Additional functional variants of antibodies that can be used as antibody therapeutic agents include antibody-like molecules other than antigen binding-Fc receptor binding domain fusions. For example, antibodies, functional fragments and fusions thereof containing a Fc receptor binding domain can be produced to be bispecific in that one variable region binding domain exhibits binding activity for one antigen and the other variable region binding domain exhibits binding activity for a second antigen. Such bispecific antibodies can be advantageous in the methods of the invention because a single bispecific antibody will contain two different target antigen binding species. Therefore, a single molecular entity can be administered to achieve neutralization of, for example, both IP-10 and another chemokine. Such antibodies are also considered “anti-IP-10 antibodies” in this disclosure.

An “anti-IP-10 antibody” can also be an immunoadhesion or bispecific immunoadhesion. Immunoadhesions are antibody-like molecules that combine the binding domain of a non-antibody polypeptide with the effector functions of an antibody of an antibody constant domain. The binding domain of the non-antibody polypeptide can be, for example, a ligand or a cell surface receptor having ligand binding activity. Immunoadhesions for use as anti-IP-10 antibodies can contain at least the Fc receptor binding effector functions of the antibody constant domain. Specific examples of ligands and cell surface receptors that can be used for the antigen binding domain of an immunoadhesion therapeutic agent include, for example, a T cell or NK cell receptor, such as the CXCR3 receptor that recognizes IP-10.

Other ligands and ligand receptors known in the art can similarly be used for the antigen binding domain of an immunoadhesion anti-IP-10 antibody. In addition, multivalent and multispecific immunoadhesions can be constructed for use as anti-IP-10 antibodies. The construction of bispecific antibodies, immunoadhesions, bispecific immunoadhesions and other heteromultimeric polypeptides which could be used according to this invention as IP-10 specific antibodies is the subject matter of, for example, U.S. Pat. Nos. 5,807,706 and 5,428,130, which are incorporated herein by reference.

Antibody therapeutic agents which have been approved for marketing include Orthoclone OKT3® (Johnson & Johnson), ReoPro® (Lilly), Rituxan® (Genentech), Simulect® (Novartis), Remicade® (Johnson & Johnson), Zenapax® (Roche), Synagis® (Medimmune), Herceptin® (Genentech), Mylotarg® (American Home Products), Campath® (Millennium), Zevalin® (IDEC Pharmaceuticals and Schering AG), and Humira® (Abbott Laboratories). These agents use various antibody formats including murine, chimeric, CDR-grafted, radiolabeled and phage display. Current strategies for antibody development by leading companies such as Abgenix, Inc. (Freemont, Calif.), Protein Design Labs, Inc. (Fremont, Calif.), and Medarex, Inc. (Princeton, N.J.) emphasize the preference for humanized antibody formats.

Use of Anti-IP-10 Antibodies to Treat Rheumatoid Arthritis

Successful treatment of RA in humans with humanized anti-IP-10 antibody (or combination therapy with humanized anti-IP-10 antibody in combination with anti-RA therapeutic compound candidates) requires proper dosing and administration of the compound, as is well understood by those skilled in the art. A variety of proposed dosage regimens are provided below. It is within the skill of the art to select an appropriate dosing regimen that will optimize safety and efficacy. The humanized anti-IP-10 antibody will be administered in a dose sufficient to treat RA in an individual. An amount of anti-IP-10 antibody adequate to accomplish this is defined as a “therapeutically effective” dose.

Therapeutically effective doses of anti-IP-10 antibody will vary depending upon many different factors, including means of administration, target site, physiological state of the patient, judgment of the physician, and interaction with the other anti-RA therapeutic compound candidates. Similarly, therapeutically effective doses of anti-RA therapeutic compound candidates will vary depending upon many factors, including means of administration, physiological state of the patient, judgment of the physician and interaction with humanized anti-IP-10 antibodies. As used herein, “anti-RA therapeutic compound candidates” refers to therapeutic compounds useful for treating RA. Examples of anti-RA therapeutic compound candidates useful in the present invention are listed in Tables 1 and 2. In embodiments that employ combination therapy, at least one anti-RA therapeutic compound candidate is used together with the humanized anti-IP-10 antibody of the present invention to form the combination therapy of the present invention.

In general, therapeutically effective doses of anti-IP-10 antibody range from about 0.0001 to about 100 milligrams of antibody per kilogram body weight of individual to whom it is administered (mg/kg), more usually from about 1 to about 15 mg/kg, preferably from about 5 to about 10 mg/kg and most preferably about 8 mg/kg. Accordingly, given an average human weighing 70 kg, a therapeutically effective dose of anti-IP-10 antibody ranges from about 0.0070 mg to about 7000 mg, more usually from about 70 mg to about 1050 mg, preferably from about 350 mg to about 700 mg, and most preferably about 560 mg per dose.

Pharmaceutical compositions useful in the present invention comprising anti-IP-10 antibody may include a pharmaceutically acceptable carrier for the antibody. The pharmaceutical compositions useful in the present invention may be administered by parenteral, topical, oral, intravenous, subcutaneous, intrathecal, intramuscular or other local delivery technique. Preferred techniques will allow the anti-IP-10 antibody suitable access to the joint or disease area where IP-10 accumulates to provide the combination maximum effect on the individual. In one embodiment, the humanized anti-IP-10 antibody of the present invention may be formulated in an aqueous carrier according to techniques well known to those of skill in the art, which techniques may include formulation in, for example, water, buffered water, saline, glycine, hyaluronic acid, and the like. Such aqueous compositions may be sterilized by conventional methods known to those of skill in the art or, alternatively, may be sterile filtered. Additionally, the aqueous solutions of the present invention may be packaged for use as is or may be lyophilized to be reconstituted with sterile solution prior to administration. It is to be understood that aqueous solutions may contain pharmaceutically acceptable excipients, such as, for example, buffering agents, tonicity adjusting agents, wetting agents and the like.

Those skilled in the art are aware that preferred dosing may be daily, every 2, 3, 4, 5 or 6 days, weekly, every 2, 3, 4, 5, 6, 7, or 8 weeks, or monthly, or every 2, 3, 4, 5, or 6 months. Because of the long circulating half-life of humanized anti-IP-10 antibodies, preferred dosing may be at least 2 days, at least 5 days, at least 1 week, at least 2 weeks, at least 3 weeks, at least 4 weeks, at least 6 weeks or at least 8 weeks. Moreover, intravenous dosing of about once per month may be preferred to maintain sufficiently high levels of humanized anti-IP-10 antibody to treat RA. More regular dosing of humanized anti-IP-10 antibody to treat RA, if necessary, will preferably employ an intramuscular administration.

Some embodiments of the present invention contemplate use of humanized anti-IP10 antibodies in combination with anti-RA therapeutic compound candidates. Many therapeutic compounds are currently in use or in development to treat RA. In this specification, such compounds are called “anti-RA therapeutic compound candidates.” In certain embodiments of the present invention, anti-IP-10 antibodies maybe used in combination with at least one other anti-RA therapeutic compound candidate. Examples of anti-RA therapeutic compound candidates are listed in Tables 1 and 2.

TABLE 1
Anti-RA therapeutic compound candidates approved for marketing.

| | Exemplary Trade | | | |
|---|---|---|---|---|
| Compound | Name(s) | Vendor | Target | Dosing |
| Anti-Inflammatory | | | | |
| Aspirin and Non- | Ibuprofen (Motrin, | Various | Anti- | |
| Steroidal Anti- | Advil), Fenoprofen, | | inflammatory | |
| Inflammatory Drugs | Indomethacin, | | agents | |
| (NSAIDS) | Naproxen (Naprosyn) | | | |
| Coxibs | Rofecoxib (Vioxx) and | Merck | Cox-2 inhibitors | |
| | Etoricoxib (Arcoxia) | Pfizer | | |
| | Celecoxib (Celebrex), | | | |
| | Bextra | | | |
| Corticosteroids | various | various | Anti- | use is limited |
| | | | inflammatory | to short |
| | | | | courses and |
| | | | | low doses |
| | | | | where |
| | | | | possible |
| Disease-Modifying | | | | |
| Anti-Rheumatic | | | | |
| Drugs (DMARDs) | | | | |
| Methotrexate | Rheumatrex | | | |
| Injectable gold | Myochrysine, Solganal | | | |
| Oral gold | Auranofin/Ridaura | | | |
| Hydroxychloroquine | Plaquenil | | | usually in |
| | | | | conjunction |
| | | | | with |
| | | | | Methotrexate. |
| Sulfasalazine | Azulfidine | | | usually in |
| | | | | conjunction |
| | | | | with |
| | | | | Methotrexate. |
| Biologicals | | | | |
| Etanercept | Enbrel | Amgen | Fully | One |
| | | (Immunex) | humanized | subcutaneous |
| | | Wyeth | tumor necrosis | injection twice |
| | | | factor (TNF) | weekly. |
| | | | inhibitor. | |
| Infliximab | Remicade | J&J/ | anti-TNF | Intravenous |
| | | Schering- | antibody (not | infusion every |
| | | Plough | fully | two months |
| | | | humanized) | (after 3 initial |
| | | | | injections). 3-10 mg/kg. |
| | | | | Dosed in |
| | | | | combination |
| | | | | with |
| | | | | methotrexate |
| Leflunomide | Arava | Aventis | blocks the | |
| | | | growth of new | |
| | | | cells | |
| Anakinra | Kineret | Amgen | (IL-1 RA) | One |
| | | | Interleukin-1 | subcutaneous |
| | | | receptor | injection daily |
| | | | antagonist | |
| Immune | | | | |
| Suppressors | | | | |
| Azathioprine | Imuran | | | associated |
| | | | | with toxic side |
| | | | | effects, |
| | | | | reserved for |
| | | | | severe cases |
| | | | | of RA |
| Cyclophosphamide | Cytoxan | | | associated |
| | | | | with toxic side |
| | | | | effects, |
| | | | | reserved for |
| | | | | severe cases |
| | | | | of RA |

The anti-RA therapeutic compounds of Table 1 may be employed in combination with humanized anti-IP-10 antibody according to the methods of the present invention.

TABLE 2
Anti-RA therapeutic compounds awaiting marketing approval.

| Compound | Target | Dosing | Sponsor |
|---|---|---|---|
| TA-650 | | | Tanabe |
| Adalimumab | Anti-TNF mAB | Sub- | Abbott |
| (D2E7) | | cutaneous | Laboratories |
| | | injection | |
| | | once every | |
| | | two weeks | |
| | | or infusion | |
| | | (3-5 minute | |
| | | IV push) | |
| Prexige | Cox2 Inhibitor | | Novartis |
| CDP-870 | PEG-antibody anti- | once | Celltech/Pfizer |
| | TNF therapy | monthly | |
| | | dosing | |
| BMS-188667 | B7 receptor, | 2 mg/kg | Bristol-Meyers |
| (CTLA4lg) | prevents T-cell and | Likely for | Squibb (BMS) |
| | cytokine production | use in | |
| | | combination | |
| | | with anti- | |
| | | TNF | |
| | | therapies | |
| YM-177 | Cox2 Inhibitor | | Yamanouchi |
| ISIS 104838 | TNF inhibitor | | Isis |
| antisense | | | Pharmaceuticals |
| Paxceed | Microtubule | | Angiotech |
| (paclitaxel) | inhibitor | | Pharmaceuticals |
| J-695 | Anti-IL-12 mAb | | Abbott |
| ZD 2315 | Immunomodulator | | AstraZeneca |
| Tillmacoxib | Cox2 inhibitor | | Fujisawa |
| (JTE-522) | | | |
| Mabthera | | | Roche/Genentech/ |
| | | | IDEC |
| DPC-168 | CCR3 (Eotaxin) | | BMS |
| | Antagonist | | |
| DPC-333 | TNFa Converting | DMARD | BMS |
| | Enzyme (TACE) | | |
| | Inhibitor | | |
| MRA | Anti-IL6 receptor | | Chugai |
| | mAb | | |
| CS-502 | Cox2 Inhibitor | | Sankyo |
| ABT-963 | Cox2 Inhibitor | | Abbott |
| GW406381 | Cox2 Inhibitor | | GlaxoSmithKline |
| | | | (GSK) |
| SB 273005 | Osteoclast | | GSK |
| | vitronectin receptor | | |
| | antagonist | | |
| SB 281832 | p38 kinase inhibitor | | GSK |
| R1487 | | | Roche |
| TR-14035 | Dual alpha4 integrin | | Tanabe |
| | antagonist (VLA4) | | |
| PLA-725/PLA- | acts upstream of | | Wyeth |
| 902 | coxibs | | |
| Efalizumab | | | Xoma |
| MRA mAb | blocks the action of | | NIAMS |
| | interleukin-6 (IL-6), | | |
| AZD7140 | chemokine receptor | | AstraZeneca |
| | antagonist | | |
| AZD8309 | chemokine receptor | | AstraZeneca |
| | antagonist | | |
| AZD9056 | Ion channel blocker | | AstraZeneca |
| DPC A37818 | CCR3 (Eotaxin) | | BMS |
| R-125224 | anti-fas mAb | | Sankyo |
| SSR 150106 | TNF alpha and | | Sanofi-Synthelabo |
| | MCP1 inhibitor | | |
| R-132811 | p38 MAP Kinase | | Sankyo |
| | inhibitor | | |

Anti-IP-10 antibody may also be used in combination with the anti-RA therapeutic compound candidates in Table 2 according to the methods of the present invention.

Preferred Combination Therapies

While not wishing to be bound to any specific mechanism of action, it is believed that anti-IP-10 antibody treats RA by removing or reducing the chemokine IP-10 from the vicinity of synovial tissue or inflamed tissue, thereby preventing or reducing the accumulation of T-cells and other cells of the immune system, which are believed to be responsible for chronic and persistent damage to synovial tissues. Moreover, anti-IP-10 antibodies may result in a treatment having reduced side effects as compared to current RA treatments that target the CXCR3 receptor. By targeting IP-10, which is only one of several known ligands of the CXCR3 receptor, the methods of the present invention selectively inhibit the action of IP-10 and leave the CXCR3 receptor free to interact with its other natural ligands.

The most effective combination therapies are likely those that employ therapeutic mechanisms of action for RA management that are complementary to the mechanism of action of anti-IP-10 antibodies. Thus, aspirin and NSAIDs, such as, for example, Naprosyn® and Motrin®, as well as anti-TNF antibody, such as, for example, Infliximab, in combination with an anti-IP-10 antibody provide complementary and synergistic effects.

In another embodiment, the present invention contemplates the humanized anti-IP-10 antibodies and anti-RA therapeutic compound candidates are packaged in kits. The kits of the present invention may comprise one or more containers filled with aqueous solutions of humanized anti-IP-10 antibodies separately or together with a particular anti-RA therapeutic compound candidate. For example, a kit of the present invention may comprise one vial of humanized anti-IP-10 antibody and a separate vial comprising a therapeutically effective amount of a DMARD or a biological anti-rheumatic. Alternatively, the kit may comprise a single vial containing therapeutically effective amounts of humanized anti-IP-10 antibodies together with a therapeutically effective amount of a combination therapy candidate, such as, for example, a DMARD or a biological anti-rheumatic. A kit of the present invention may optionally include means for administering the combination therapy to an individual in need thereof, as well as instructions for proper dosing use.

RA treatment regimens comprising combination therapy likely will be most enhanced by approximately monthly dosing of humanized anti-IP-10 antibody, although other dosing schedules are possible. Preferred combinations, which are not intended to limit the scope of options set out herein, include:

| | |
|---|---|
| Enbrel ® | Twice Weekly/intramuscular/25 mg |
| Anti-IP-10 antibody | Monthly/intravenous/5-10 mg/kg |
| Remicade ® | Every 2 months/intravenous/5 mg/kg |
| Methotrexate | Weekly/oral/7.5-20 mg |
| Anti-IP-10 Antibody | Monthly/intravenous/5-10 mg/kg |

The following examples are offered to illustrate various aspects of the invention and are not to be construed as to limit the invention in any way.

### EXAMPLES

### Example 1

### Generation of Humanized Anti-IP-10 Antibody

Humanized anti-IP-10 antibodies may be generated by selecting an appropriate immunogen comprising a nucleotide sequence of IP-10 and injecting that immunogen into the HuMAb-Mouse® (Medarex, Inc.) to subsequently generate monoclonal antibodies directed against IP-10 protein.

### Example 2

### Anti-IP-10 Treatment Reduced Inflammation in the Type II Collagen-Induced Arthritis Model

Experimental results demonstrate the impressive effectiveness of anti-IP-10 antibody treatment using in the Type II Collagen-induced arthritis mouse model. This model is considered highly reflective of human inflammatory conditions, such as RA.

DBA Lac/J mice (7 week old (Jackson Labs cat# 001140)) (n=20) were injected intradermally (i.d. 100 ul) at the base of the tail. with a 1:1 mixture containing 2 mg/ml Type II Collagen (CII) (Sigma C-9301) dissolved in 0.01 M Acetic Acid (Fisher A38-500) and incomplete Freund's adjuvant (IFA) (Sigma F-5506)+M. tuberculosis H37RA (Difco DF3114-33-8) 4 mg/ml. An i.d. booster injection (100 ul) of a 1:1 mixture of CII+PBS was administered at 21 days post-induction. A final injection of LPS (Cal Biochem 437620) was administered intraperitoneally at day 28 post-induction. Mice were separated into 5 groups. All treatments were administered intraperitoneally, with the following treatment schedules: 1) no treatment (positive control); 2) treatment with 0.5 mg anti-RANTES antibody (Biosynthesis mono-specific polyclonal antibody) immediately upon appearance of paw swelling; 3) treatment with 0.5 mg anti-RANTES antibody 7 days following appearance of paw swelling; 4) treatment with 0.5 mg anti-IP-10 antibody (monoclonal IP6C7 antibody prepared by Ability Biomedical Corporation, Irvine, Calif.) immediately upon appearance of paw swelling; and 5) treatment with 0.5 mg anti-IP-10 antibody 7 days following appearance of paw swelling. All experiments were conducted according to protocols approved by the relevant animal care committee.

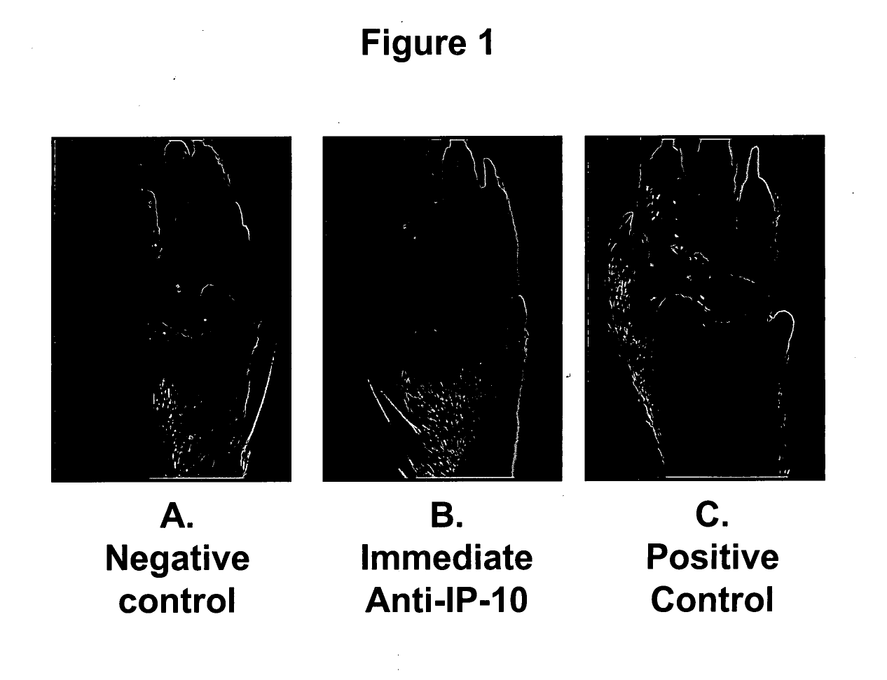

As shown in FIG. 1, early treatment with anti-IP-10 antibody remarkably diminished paw swelling. Representative samples of paws are shown after treatment.

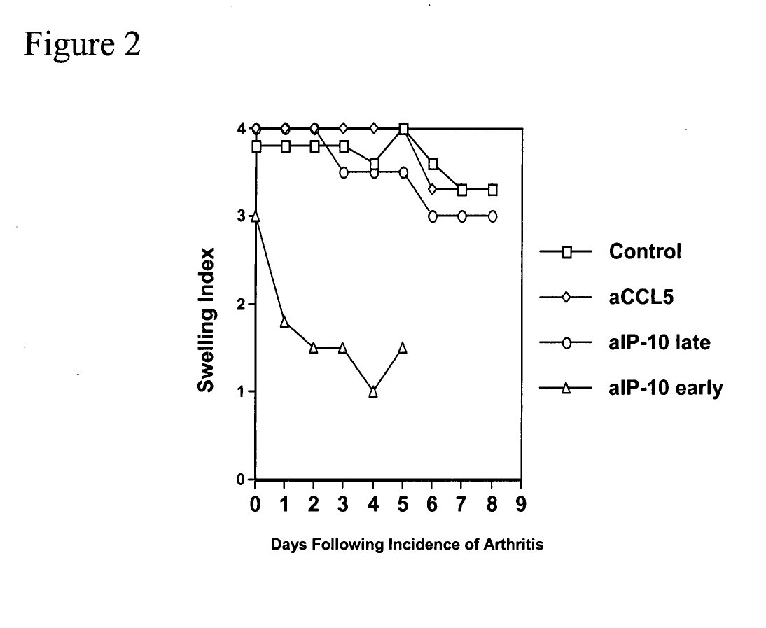

Inflammation was measured by visual inspection of the injected paw. FIG. 2 records measurements of inflammation in the days following incidence of arthritis. The following scale was used to score evidence of inflammatory activity: 0=No evidence of erythema and swelling; 1=Erythema and mild swelling confined to the mid-foot (tarsals) or ankle joint; 2=Erythema and mild swelling extending from the ankle to the mid-foot; 3=Erythema and moderate swelling extending from ankle to metatarsal joints; 4=Erythema and severe swelling encompass the ankle, foot, and digits.

As shown in FIG. 2, early anti-IP-10 treatment provides a substantial reduction in inflammation compared to control. Anti-RANTES (CCL5) (early or late) treatment provided no significant difference from controls. Late treatment with anti-IP-10 antibodies provided only a modest reduction in inflammation compared to controls.

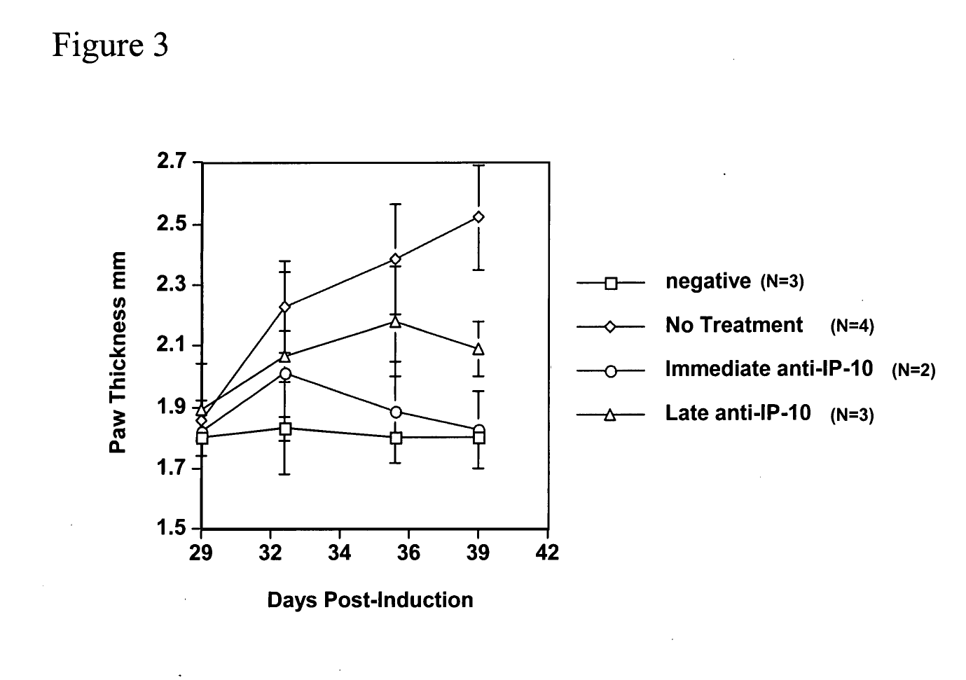

FIG. 3 records measurements of paw swelling (in millimeters, m). Again these direct measurements of inflammatory activity showed that early treatment with anti-IP-10 provided a substantial reduction in paw thickness, while late treatment provided a measurable, but less effective reduction in paw thickness. Anti-RANTES treatment showed no difference from controls.

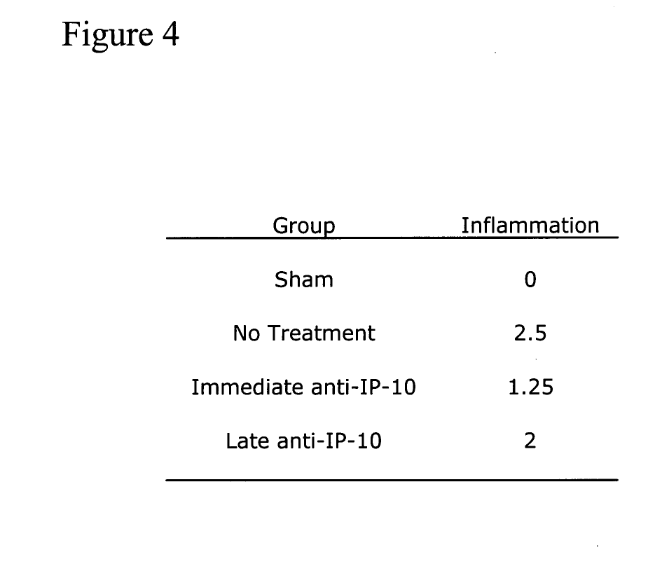

At day 39 post induction of inflammation, mice were sacrificed, and histological sections of injected paws were prepared. FIG. 4 records histological scoring of inflammation on the following scale: 0=No evidence of disease; 1=Mild lymphocytic infiltrate; 2=Widespread mononuclear inflammation and thickening of the synovial lining; 3=Severe bone destruction, new bone formation, and destruction of the synovial lining. Results show that early treatment with anti-IP-10 antibody had the most substantial effect on reduction of inflammation, followed by late treatment with anti-IP-10 antibody, followed by no treatment (positive) control.

### Example 3

### Combination Therapy Using Anti-IP-10 Antibody in Treatment of Type II Collagen Induced Arthritis Model

The synergistic benefit of treatment with anti-IP-10 antibody in combination with a complementary anti-RA treatment, such as anti-TNF-alpha antibody, is demonstrated as follows. A mouse Type II Collagen-induced arthritis model is prepared as previously described. Control animals are prepared as previously described. Mouse specific anti-TNF-alpha antibody (preferably monoclonal) is prepared in a mouse by means well known in the art. Mouse specific anti-IP-10 antibody is prepared as previously described. Test mice are treated with 5-10 mg/kg of each antibody separately, and in combination, at various doses. Combination therapy treatments demonstrate an improved synergistic response compared to each treatment individually.

The present invention may be embodied in other specific forms without departing from the teachings or essential characteristics of the invention. The described embodiments are therefore to be considered in all respects as illustrative and not restrictive, the scope of the invention being indicated by the appended claims rather than the foregoing description, and all changes that come within the meaning and range of equivalency of the claims are to be embraced within.

1. A method for treating rheumatoid arthritis in an individual, the method comprising administering to the individual a therapeutically effective amount of an anti-IP-10 antibody.

2. The method of claim 1, wherein the anti-IP-10 antibody is a humanized antibody.

3. The method of claim 1, wherein the anti-IP-10 antibody is a human antibody.

4. The method of claim 1, wherein the therapeutically effective amount of humanized anti-IP-10 antibody is administered in a dose ranging from about 1 mg/kg to about 15 mg/kg.

5. The method of claim 1, wherein the therapeutically effective amount of humanized anti-IP-10 antibody is administered in a dose ranging from about 5 mg/kg to about 10 mg/kg.

6. The method of claim 1, wherein the therapeutically effective amount of humanized anti-IP-10 antibody is administered in a dose of about 8 mg/kg.

7. The method of claim 1, wherein the therapeutically effective amount of humanized anti-IP-10 antibody is administered about once every 3 weeks.

8. The method of claim 1, wherein the therapeutically effective amount of humanized anti-IP-10 antibody is administered about once every month.

9. A method for treating rheumatoid arthritis in an individual, the method comprising administering to the individual a therapeutically effective amount of an anti-IP-10 antibody in combination with a therapeutically effective amount of a compound selected from the group consisting of anti-inflammatory agents, disease-modifying-anti-rheumatic drugs, anti-rheumatic biologicals, immune suppressors, and the compounds listed in Table 2.

10. The method of claim 9, wherein the anti-inflammatory agents are selected from the group consisting of aspirin, NSAIDS, coxibs, and corticosteroids.

11. The method of claim 9, wherein the disease-modifying-anti-rheumatic drugs are selected from the group consisting of methotrexate, injectable gold, oral gold, hydroxychloroquine, and sulfasalazine.

12. The method of claim 9, wherein the anti-rheumatic biologicals are selected from the group consisting of etanercept, infliximab, leflunomide, and anakinra.

13. The method of claim 9, wherein the immune suppressors are selected from the group consisting of azathioprine and cyclophosphamide.

14. The method of claim 9, wherein the anti-IP-10 antibody is a humanized antibody.

15. The method of claim 9, wherein the anti-IP-10 antibody is a human antibody.

16. A method for treating rheumatoid arthritis in an individual, the method comprising:
administering to the individual a therapeutically effective amount of an anti-IP-10 antibody in combination with a therapeutically effective amount of a compound selected from the group consisting of anti-inflammatory agents, disease-modifying-anti-rheumatic drugs, anti-rheumatic biologicals, immune suppressors, and the compounds listed in Table 2; and
determining whether the individual has been successfully treated.

17. The method of claim 16, wherein the anti-IP-10 antibody is a humanized antibody.

18. The method of claim 16, wherein the anti-IP-10 antibody is a human antibody.

19. The method of claim 16, wherein the anti-inflammatory agents are selected from the group consisting of aspirin, NSAIDS, coxibs, and corticosteroids.

20. The method of claim 16, wherein the disease-modifying-anti-rheumatic drugs are selected from the group consisting of methotrexate, injectable gold, oral gold, hydroxychloroquine, and sulfasalazine.

21. The method of claim 16, wherein the anti-rheumatic biologicals are selected from the group consisting of etanercept, infliximab, leflunomide, and anakinra.

22. The method of claim 16, wherein the immune suppressors are selected from the group consisting of azathioprine and cyclophosphamide.

23. The method of claim 16, wherein the step of determining whether rheumatoid arthritis has been successfully treated comprises:
subjecting the individual to a test that measures an arrest, decrease, or reversal in signs, physiological indicators, biochemical markers, or metabolic indicators associated with rheumatoid arthritis, wherein the test is selected from the group consisting of measurements of symmetric joint swelling, fusiform swelling, pain, fatigue, morning stiffness (lasting more than one hour), diffuse muscular aches, loss of appetite, weakness, joint pain with warmth, swelling, tenderness, and stiffness of a joint after inactivity.

24. A kit useful for treating rheumatoid arthritis in an individual, the kit comprising:
a therapeutically effective amount of a humanized anti-IP-10 antibody;
a therapeutically effective amount of a compound selected from the group consisting of anti-inflammatory agents, disease-modifying-anti-rheumatic drugs, anti-rheumatic biologicals, immune suppressors, and the compounds listed in Table 2;
means for administering the antibody and the compound in combination with each other; and
instructions for determining proper dosing and administration of the combination to the individual.
